(19) United States
(12) Patent Application Publication
(10) Pub. No.: US 2024/0189541 A1
(43) Pub. Date: Jun. 13, 2024

(54) Tactile Calming Device

(71) Applicant: Roy Steinberg, Highland Park, IL (US)

(72) Inventor: Roy Steinberg, Highland Park, IL (US)

(21) Appl. No.: 18/482,595

(22) Filed: Oct. 6, 2023

Related U.S. Application Data
(60) Provisional application No. 63/432,148, filed on Dec. 13, 2022.

(51) Int. Cl. A61M 21/02

(52) U.S. Cl. CPC A61M 21/02; A61M 2021/0022

(57) ABSTRACT

A tactile calming apparatus has a base, a vertical column extending orthogonally from the base, the vertical column having indented tracks on opposite sides along the height of the column, a carriage riding on bearings in the indented tracks, and a spring adapted to provide a force against the carriage in a direction away from the base. A user grasps the apparatus by a thumb of one hand under the base and fingers over the carriage and manipulates the apparatus by repetitive pinching motions between the thumb and fingers.

### CROSS-REFERENCE TO RELATED APPLICATIONS

The instant application claims priority to Provisional Patent Application 63/432,148, filed Dec. 13, 2022. All disclosure of the parent application is incorporated at least by reference.

### BACKGROUND OF THE INVENTION

### 1. Field of the Invention

The present invention is in the field of therapeutic tactile apparatus and pertains particularly to an apparatus for calming an individual from restlessness and fidgeting.

### 2. Discussion of the State of the Art

In therapeutic art, treatments for individuals who suffer from restlessness caused by various conditions, such as attention deficit disorder (ADD), attention deficit hyperactive disorder (ADHD), and autism spectrum disorder (ASD) and other similar conditions may include apparatus that may be manipulated to provide a harmless and focused activity to help calm the person. In current art, fidget cubes, fidget spinners, and similar manipulable apparatus are available but may be over stimulating, under stimulating and distracting to a user and may also be distracting to other persons present due to noise made using the apparatus. Stress balls and similar objects are also available but do not provide the sort of tactile experience that provides both a tactile sensation and calming sound in a manner that is not a distraction to other persons in the presence of the user.

Therefore, what is clearly needed is a therapeutic tactile apparatus that has a natural movement that overcomes the limitations described above.

### BRIEF SUMMARY OF THE INVENTION

In one embodiment of the invention a tactile calming apparatus is provided, comprising a base, a vertical column extending orthogonally from the base, the vertical column having indented tracks on opposite sides along the height of the column, a carriage riding on bearings in the indented tracks, and a spring adapted to provide a force against the carriage in a direction away from the base. A user grasps the apparatus by a thumb of one hand under the base and fingers over the carriage and manipulates the apparatus by repetitive pinching motions between the thumb and fingers.

In one embodiment the spring is a tension spring tethered at one end at an upper region of the column above the carriage, and at an opposite end to the carriage. Also, in one embodiment the spring is a compression spring positioned between the base and the carriage providing a spring force against the carriage directed away from the base. In one embodiment the bearings are roller bearings pinned to the carriage on opposite sides positioned in the tracks, and are sized to guide closely in the tracks, preventing excessive side-to-side motion. And in one embodiment the base is circular ergonomic cushioned platform rounded at the top and bottom edges for comfort.

In one embodiment the underside of the base is shaped to a user's thumb. Also, in one embodiment the carriage has wings on opposite sides shaped to slope downward away from the column for a user's fingers. Also, in one embodiment the wings are radiused generally for a user's finger tips to prevent the fingers from slipping off the carriage. In one embodiment the apparatus further comprises a tether interface at an upper region of the column adapted to connect the apparatus to a lanyard, a key ring, a necklace, or other personal item of a user to ensure the apparatus is always at hand when needed. And in one embodiment the tracks have cross ribs molded in a surface of the tracks, such that the bearings rolling over the cross ribs produce a soft sound and feel.

In another aspect of the invention a method for calming an individual from restlessness and fidgeting is provided, comprising providing a tactile calming apparatus having a base, a vertical column extending orthogonally from the base, the vertical column having indented tracks on opposite sides along the height of the column, a carriage riding on bearings in the indented tracks, and a spring adapted to provide a force against the carriage in a direction away from the base, and grasping the apparatus by a user by a thumb of one hand under the base and fingers over the carriage and manipulating the apparatus by repetitive pinching motions between the thumb and fingers.

In one embodiment of the method the spring is a tension spring tethered at one end at an upper region of the column above the carriage, and at an opposite end to the carriage, comprising creating a spring force between the column and the carriage. In one embodiment of the method the spring is a compression spring positioned between the base and the carriage, comprising creating a spring force against the carriage directed away from the base. In one the bearings are roller bearings pinned to the carriage on opposite sides positioned in the tracks, comprising sizing the bearings to guide closely in the tracks, preventing excessive side-to-side motion. And in one embodiment the method comprises making the base as a circular ergonomic cushioned platform rounded at the top and bottom edges for comfort.

In one embodiment the method comprises shaping the underside of the base to a user's thumb. Also, in one embodiment the method comprises forming the carriage with wings on opposite sides shaped to slope downward away from the column for a user's fingers. Also, in one embodiment the method comprises shaping the wings generally for a user's finger tips to prevent the fingers from slipping off the carriage. In one embodiment the method further comprises forming a tether interface at an upper region of the column, the interface adapted to connect the apparatus to a lanyard, a key ring, a necklace, or other personal item of a user to ensure the apparatus is always at hand when needed. And in one embodiment the method comprises forming the tracks with cross ribs molded in a surface of the tracks, such that the bearings rolling over the cross ribs produce a soft sound and feel.

### BRIEF DESCRIPTION OF THE SEVERAL VIEWS OF THE DRAWINGS

FIG. 1 is a perspective view of an apparatus in an embodiment of the invention.

FIG. 2A is an elevation view of apparatus 100 of FIG. 1 in expanded mode.

FIG. 2B is an elevation view of the apparatus of FIG. 2A in compressed mode.

FIG. 3 is a side elevation view of apparatus 100 of FIG. 1.

FIG. 4A is an overhead view of apparatus 100 of FIG. 1.

FIG. 4B is a bottom view of apparatus 100 of FIG. 1.

### DETAILED DESCRIPTION OF THE INVENTION

In various embodiments described in enabling detail herein, the inventor provides a unique tactile calming apparatus for therapeutic manipulation by persons having a restless, nervous, or other hyper state symptom, such as ADD, ADHD, ASD, or similar conditions. The present invention is described using the following examples, which may describe more than one relevant embodiment falling within the scope of the invention.

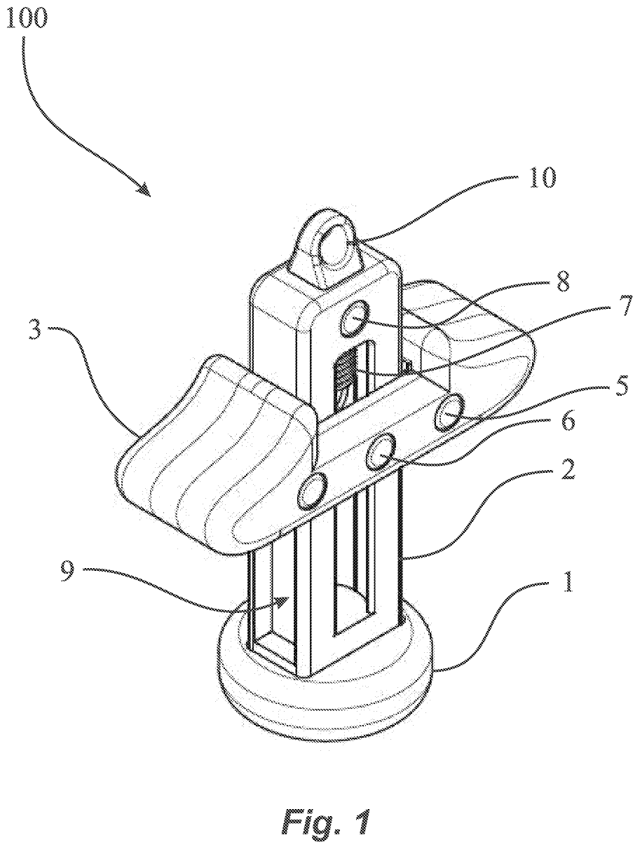

FIG. 1 is a perspective view of an apparatus 100 in one embodiment of the invention. Apparatus 100 is adapted as a manipulable mechanical assembly that may be operated by a user with one hand. Apparatus 100 comprises a base 1 with a column 2 anchored to the base and extending upward from the base. Column 2 has indented tracks 9 on opposite sides of the base. A carriage 3 is linearly translatable along the carriage by a pair of roller bearings anchored by pins 5 in the carriage and following the tracks 9.

A tension spring 7 is held at one end by a pin 8 in the column and at an opposite end by a pin 6 in the carriage. It may be seen that carriage 3 has shaped wings on opposite sides such that a user may grasp the apparatus with the carriage on the thumb of one hand and the fingers over the wings of the carriage. The user may squeeze the apparatus urging the carriage against the spring force toward base 1. The tension increases with the operation as the carriage approaches the base. Column 2, finger carriage 3, and base 1 may be molded parts made of a rigid plastic or polymer material.

In an alternative embodiment a compression spring may be used between the base and the carriage rather than the tension spring tethered to the top of the column and to the carriage. Tracks 9 in one embodiment are just wider than the thickness of the wheel bearings such that little side-to-side motion is experienced by the bearings rolling along the tracks. In one embodiment a tethering interface is provided at an upper end of column 2 such that apparatus 1 may be attached to a lanyard, a key chain, worn on a necklace, or otherwise secured to a user or personal item of a user to ensure it is always at hand when needed.

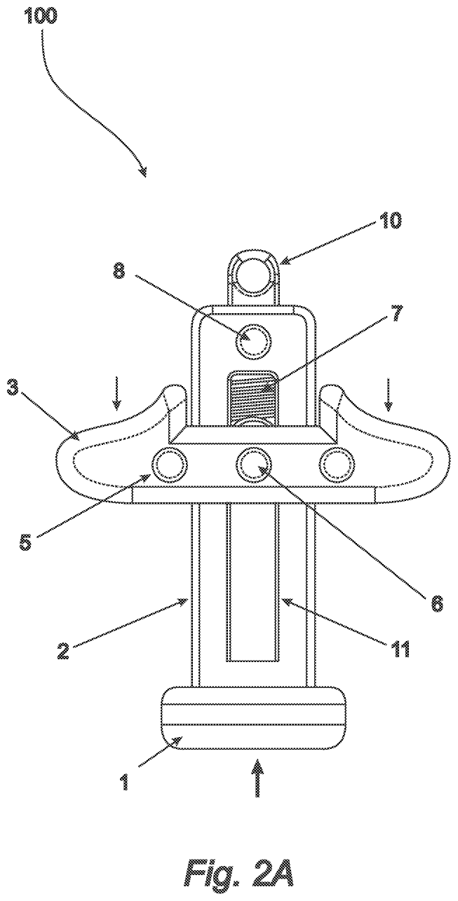

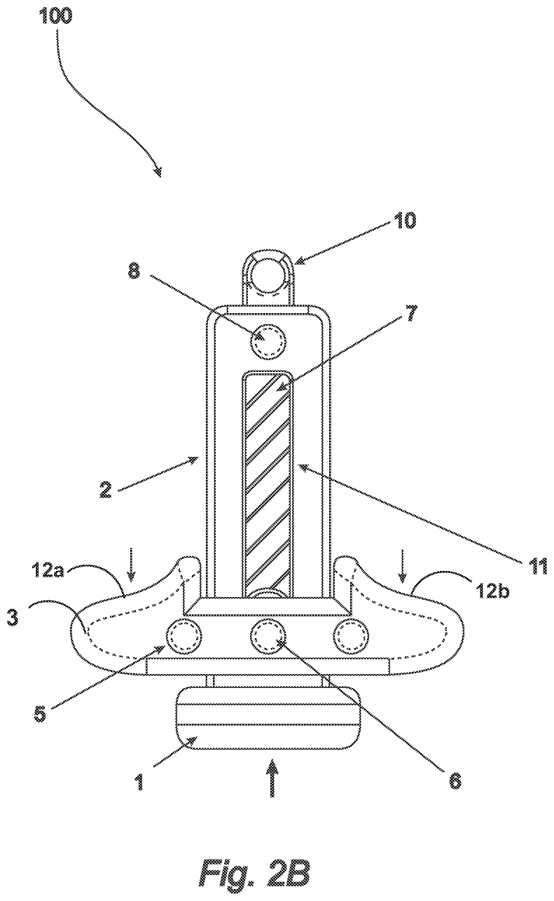

FIG. 2A is an elevation view of apparatus 100 of FIG. 1 in expanded mode with carriage 3 in a default position resting at a position near a top end of column 2. In this view, carriage 3 is seen to be formed in an ergonomic symmetrical shape with sloping finger platforms and well-rounded edges for user comfort and consistent compression from each side. Upper surfaces of carriage 3 may be radiused, providing indentations on opposing ends of the carriage generally for a user's finger tips to rest in, in order to prevent the fingers from slipping off the carriage. Spring 7 may be a steel spring with a gauge that may be suitable for the purpose of providing resistance for operation without tasking the user unnecessarily or causing pain for the user. In one embodiment, apparatus 100 may be provided with more than one spring having different gauges, for example, as deemed appropriate for different patient types (adult, child) and or patients having differing physical conditions. Alternative springs may be removed and replaced according to need.

FIG. 2B is an elevation view of apparatus 100 with the carriage 3 in a compressed position. In this view, carriage 3 also has an ergonomic symmetrical shape with sloping finger platforms 12a, 12b and well-rounded edges for user comfort and consistent compression from each side. Base 1, as a thumb pad, may be an ergonomic cushioned platform that may be rounded at the top and bottom edges thereof for comfort. In one embodiment, base 1 may be indented or radiused to cradle the user's thumb according to ergonomic design and to discourage slipping of the thumb off the base. Using a pinching motion, a user may manipulate apparatus 100 according to the directional arrows translating carriage 3 down column 2 toward base 1 against spring tension. A central opening 11 may be provided longitudinally through a center portion of column 2 extending the length of the bearing tracks 9 described further above. Opening 11 may be a framed window to provide material relief for extension of spring 7. An object of the invention is to facilitate repetitions of the pinching motion against the device in an ergonomic fashion without distracting the user and without distracting others in the presence of the user.

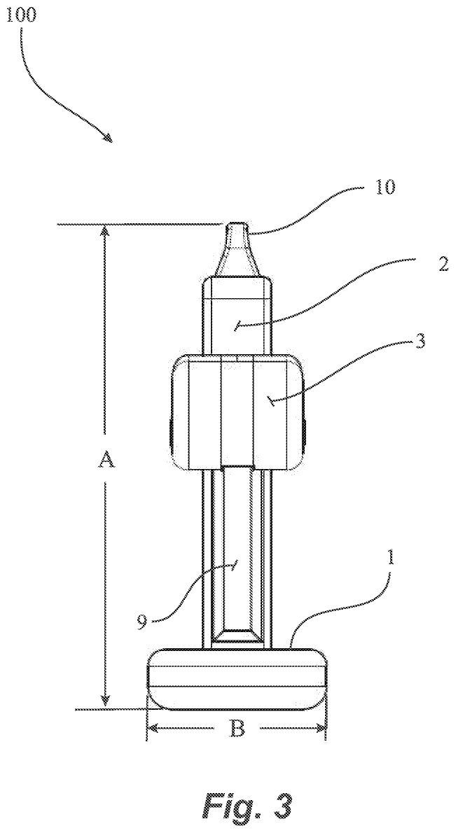

FIG. 3 is a side elevation view of apparatus 100 of FIG. 1. Apparatus 100 may generally be scaled in size and weight for different groups of users, for example, for adults or for children where the device may have a different length, width, and perhaps weight for different groups of users. In this example, an overall length dimension A of tactile calming device 100 may be about two and three-quarter inches. A width B (diameter) of base 1 of apparatus 100 may be about three-quarter inches to an inch for adult use. It is noted herein that the actual dimensions may be smaller than dimension A and dimension B for children.

In another embodiment track 9 may have cross ribs spaced along the track whereby operating the apparatus will roll the bearings over the cross ribs creating a soft washboard sound. In such an embodiment, the pinching motion and sound together may aid the user in calming and reducing restlessness. The spacing of the cross ribs may be constant or variable creating a different sort of sound and feel for the user. In still another embodiment, wheel bearings pinned to carriage 3 and positioned to roll against the bottom surfaces of track 9 may be replaced with pinned friction bearings without departing from the scope of the invention. It may be noted 30 herein that the friction bearings may have contact edges modified to click against ridges or other equally spaced features provided to the bottom surfaces of track 9 while sliding up and down column 2, making a soft sound as previously described.

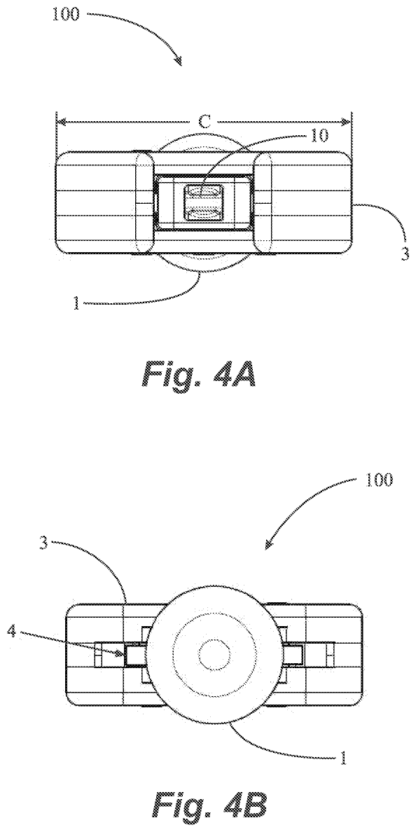

FIG. 4A is an overhead view of apparatus 100 of FIG. 1, and FIG. 4B is a bottom view of apparatus 100 of FIG. 1. Referring now to FIG. 4A, a length dimension C for carriage 3 may be about one and three-quarter inch to perhaps two inches. Finger separation distances for adults and children may be different and therefore dimension C may be smaller to facilitate a smaller natural finger separation in a child.

Referring now to FIG. 4B, wheel bearings 4 are partially visible outside of the top surfaces of column 2. Wheel bearings 4 may be about 4 mm×8 mm×3 mm chrome steel bearings. Bearing pins 5 may be formed of polyethylene terephthalate glycol (PETG) plastic. Likewise, spring anchor pins 6 and 8 may be formed of PETG plastic. In one embodiment apparatus 100 may be assembled by placing carriage 3 over column 2 and anchoring spring 7 to the top of column 2 below tether interface 10 and to the inside center of carriage 3. Bearings 4 may then be placed in position in tracks 9 and pinned to column 2 at both sides constraining finger carriage 3 onto column 2. Base 1 may be a molded part that is glued onto the bottom of column 2.

It will be apparent with skill in the art that the apparatus of the present invention may be provided using some or all the elements described herein. The arrangement of elements and functionality thereof is described in different embodiments, each of which is an implementation of the present invention. While the uses and methods are described in enabling detail herein, it is to be noted that the embodiments described are exemplary, and not limiting to the scope of the invention. Many alterations might be made in the details of the construction and the arrangement of the elements without departing from the scope of this invention.

1. A tactile calming apparatus, comprising:
a base;
a vertical column extending orthogonally from the base, the vertical column having indented tracks on opposite sides along the height of the column;
a carriage riding on bearings in the indented tracks; and
a spring adapted to provide a force against the carriage in a direction away from the base;
wherein a user is enabled to grasp the apparatus by a thumb of one hand under the base and fingers over the carriage, and manipulates the apparatus by repetitive pinching motions between the thumb and fingers reducing a distance between the base and the carriage.

2. The tactile calming apparatus of claim 1 wherein the spring is a tension spring tethered at one end at an upper region of the column above the carriage, and at an opposite end to the carriage.

3. The tactile calming apparatus of claim 1 wherein the spring is a compression spring positioned between the base and the carriage providing a spring force against the carriage directed away from the base.

4. The tactile calming apparatus of claim 1 wherein the bearings are roller bearings pinned to the carriage on opposite sides positioned in the tracks, and are sized to guide closely in the tracks, preventing excessive side-to-side motion.

5. The tactile calming apparatus of claim 1 wherein the base is circular ergonomic cushioned platform rounded at the top and bottom edges for comfort.

6. The tactile calming apparatus of claim 5 wherein the underside of the base is shaped to a user's thumb.

7. The tactile calming apparatus of claim 1 wherein the carriage has wings on opposite sides shaped to slope downward away from the column for a user's fingers.

8. The tactile calming apparatus of claim 7 wherein the wings are radiused generally for a user's finger tips to prevent the fingers from slipping off the carriage.

9. The tactile calming apparatus of claim 1 further comprising a tether interface at an upper region of the column adapted to connect the apparatus to a lanyard, a key ring, a necklace, or other personal item of a user to ensure the apparatus is always at hand when needed.

10. The tactile calming apparatus of claim 1 wherein the tracks have cross ribs molded in a surface of the tracks, such that the bearings rolling over the cross ribs produce a soft sound and feel.

11. A method for calming an individual from restlessness and fidgeting, comprising:
providing a tactile calming apparatus having a base, a vertical column extending orthogonally from the base, the vertical column having indented tracks on opposite sides along the height of the column, a carriage riding on bearings in the indented tracks, and a spring adapted to provide a force against the carriage in a direction away from the base; and
grasping the apparatus by a user by a thumb of one hand under the base and fingers over the carriage and manipulating the apparatus by repetitive pinching motions between the thumb and fingers.

12. The method of claim 11 wherein the spring is a tension spring tethered at one end at an upper region of the column above the carriage, and at an opposite end to the carriage, comprising creating a spring force between the column and the carriage.

13. The method of claim 11 wherein the spring is a compression spring positioned between the base and the carriage, comprising creating a spring force against the carriage directed away from the base.

14. The method of claim 11 wherein the bearings are roller bearings pinned to the carriage on opposite sides positioned in the tracks, comprising sizing the bearings to guide closely in the tracks, preventing excessive side-to-side motion.

15. The method of claim 11 comprising making the base as a circular ergonomic cushioned platform rounded at the top and bottom edges for comfort.

16. The method of claim 15 comprising shaping the underside of the base to a user's thumb.

17. The method of claim 11 comprising forming the carriage with wings on opposite sides shaped to slope downward away from the column for a user's fingers.

18. The method of claim 17 comprising shaping the wings generally for a user's finger tips to prevent the fingers from slipping off the carriage.

19. The method of claim 11 further comprising forming a tether interface at an upper region of the column, the interface adapted to connect the apparatus to a lanyard, a key ring, a necklace, or other personal item of a user to ensure the apparatus is always at hand when needed.

20. The method of claim 11 comprising forming the tracks with cross ribs molded in a surface of the tracks, such that the bearings rolling over the cross ribs produce a soft sound and feel.
